(19) United States
(12) Patent Application Publication
(10) Pub. No.: US 2018/0356297 A1
(43) Pub. Date: Dec. 13, 2018

(54) ARTIFICIAL HORTICULTURAL PRODUCT WITH TEMPERATURE SENSOR

(71) Applicant: EMPA Eidgenössische Materialprüfungsund Forschungsanstalt, Dübendorf (CH)

(72) Inventor: Thijs DEFRAEYE, Zürich (CH)

(73) Assignee: EMPA Eidgenössische Materialprüfungsund Forschungsanstalt, Dübendorf (CH)

(21) Appl. No.: 15/775,458

(22) PCT Filed: Nov. 11, 2016
(86) PCT No.: PCT/EP2016/077441

§ 371 Date: May 11, 2018

(30) Foreign Application Priority Data
Nov. 12, 2015 (EP) 15194320.6

(51) Int. Cl. G01K 7/42; G01K 1/02; G01N 33/02; A23B 7/05

(52) U.S. Cl. CPC G01K 7/425; G01K 1/022; G01K 2007/422; A23B 7/05; G01N 33/025

(57) ABSTRACT

An artificial produce includes a housing with at least one shell. At least one data logger for temperature measurement is placed in an area of a core of the housing and a pulp simulant is integrated at least partly in the housing of the artificial produce to show optimized simulation of thermal behavior of real produce. The form and outer surface of the at least one shell replicate the form and surface texture of the real produce simulated. The at least one shell forms at least one fluidtight chamber accessible from the outside by at least one opening which are closable with plugs. The at least one chamber is filled with the pulp simulant in the form of a gel-like filling composition comprising a water-carbohydrate mixture and a gelling agent showing similar thermal conductivity, density, heat capacity and freezing point as the pulp of the produce to be simulated.

### TECHNICAL FIELD

The present invention describes an artificial produce in form of a sensor system, comprising a housing with at least one shell, in particular with two shells, wherein at least one data logger for temperature measurement is placed in an area of a core of the housing and a pulp simulant is integrated in the housing of the artificial produce and a method for manufacturing an artificial produce in form of a sensor system, as well as a housing of an artificial produce with at least one shell, in particular two shells, wherein at least one data logger is placeable in the area of a core of the housing and a pulp simulant is integrateable at least partly in the housing.

### STATE OF THE ART

Monitoring the postharvest temperature history of fresh horticultural produce, such as fruits and vegetables, is essential to evaluate the efficacy of the fresh-produce cold chain. The reason is that temperature is the single most important parameter affecting produce quality, deterioration, ripening rate and shelf life, and is directly used to predict the latter. Rapid removal of the field heat after harvest through cooling and maintaining optimum product temperature throughout the supply chain are thus of key importance. A typical cold chain for fresh produce consists of different unit operations, including forced-air precooling, transport in refrigerated trucks or long-haul maritime transport in refrigerated containers, and long-term storage in cold rooms. For convenience, we will only address fruits in the remainder of the patent application text, but the statements hold for vegetables as well. Lowering fruit temperatures reduces respiration and transpiration rate (mass loss and shrivelling), enzyme activity but also ethylene production, so ripening rate and senescence. The impact of fruit temperature on these processes can be directly quantified, for example by the Q₁₀ quotient (based on van't Hoff's rule). The Q₁₀ quotient quantifies how much more rapidly a (decay) reaction process proceeds at a temperature Tₖ₊₁₀ which is 10° C. higher than a (lower) temperature Tₖ. For most decay processes in fruit, the reaction rate doubles or triples with each increase of 10° C. (Q₁₀≈2-3). As an example, keeping fruit at a temperature which is 10° C. colder than the normal ambient conditions typically doubles the shelf life. A critical issue here is how the fruit temperature is measured. The average fruit temperature would be most representative for the overall fruit quality state. However, this average temperature cannot be easily measured in a commercial setting.

Measurements of the internal fruit or core temperature history are essential to evaluate the efficacy of cooling strategies in several unit operations in the postharvest cold chain. In forced-air precooling, the seven-eighths cooling time (SECT) is frequently applied to assess if the fruit temperature is acceptably close to the required storage temperature, by which the precooling can be stopped and the remaining heat load can be removed with less energy costs. The SECT is the time required to reduce the temperature difference between the fruit (core) pulp and the cooling air by seven eighths.

Also after the fruit is cooled down, its pulp temperature can still vary during transport in refrigerated containers or storage in cool rooms, due to intermittent operation of cooling fans and different set temperatures for each unit operation. Fruit core temperature measurements are used by governmental organisations (U.S. Department of Agriculture—USDA, Perishable Products Export Control Board—PPECB) to decide upon the acceptability of the cargo after overseas transport in refrigerated containers, for example with respect to the cold disinfestation efficacy for pests (e.g. fruit fly, false codling moth). The fruit cooling rate is also a major design criterion in the development of new ventilated packaging designs. Fruit core temperature is also an essential indicator of hot spots for commodities with a high respiration rate, such as bananas. Such hot spots can induce spontaneous ripening of the cargo during transport and should be avoided.

Next to core temperatures, fruit surface temperature and humidity measurements are used to assess the risk of surface condensation and microbial activity.

Despite the importance of fruit pulp temperature information, current industrial practice and R&D only rely to a limited extent on such measurements. As a result, the heterogeneity of fruit cooling rates, thus fruit quality, is rarely picked up in commercial cold chain operations due to the limited amount of sensors installed, for example only a few per refrigerated container. Such heterogeneity is however present at various scales: inside a box of fruit, between boxes stacked on a pallet, and between different pallets/palloxes in a container cargo or a storage room. Furthermore, measurements tracking the fruit pulp temperature throughout its entire cold chain are rare, particularly for long (overseas) chains. Academic studies have targeted several of these aspects, but the used test setups are time-consuming to install and require specialized equipment and skilled personnel, including for data processing and interpretation.

The aforementioned limitations are strongly linked to restrictions with the current measurement technology and practices for measuring fruit pulp temperature by which they are measured to a much lesser extent in commercial applications.

Different systems for monitoring of long cold chain operations are used. For example, wired sensors, such as thermocouples or point probes, have been placed inside the core of real fruit. Such wiring is intrusive, requires cabling and a connection to an external data logger. Such data loggers are quite large by which they are difficult to pack together with fresh produce. Most of them also disturb the airflow and fruit cooling conditions since they have a different size, shape and thermal behaviour as real fruit. As such they are quite intrusive. Another example are wireless, self-powered data loggers with a built-in sensor, such as iButtons®. They have been used to measure core temperatures of real fruit by placing them inside the fruit core by making an incision.

However, installing these systems in different pallets and monitoring temperatures throughout the entire chain is cumbersome and labour/time intensive. In a commercial setting, only a few measurements per cargo are performed. In addition, inserting a sensor into the fruit pulp is a quite intrusive practice and wounds the fruit.

A metabolic response will be induced, leading to additional moisture loss, enzymatic reactions and microbial activity, by which fruit will decay. The resulting biological reactions can also cross-contaminate other fruit in the package. This practice does not allow monitoring of long cold chain operations or throughout an entire chain. Finally, the biological variability between different fruits (size, shape) will make that the readings will differ a bit depending on which fruit the sensor was inserted in.

Recently the heat flow through a product can be simulated by a thermal measuring device with integrated sensors as disclosed in GB2405477. Such a thermal measuring device, comprising a housing, a pair of sensors, which may be separated by a simulant material in form of a fixed mass, is shown. Such sensor system is able to measure and record actual temperature data. The disclosed simple formed produce sensor system could so far not lead to desired results. Due to the setup, no realistic core temperature can be measured, only the heat flow between two sensors, as the thermal mass only comprises a limited part of the housing.

To reach more exact temperature measurements during transport and storage of real produce, in WO2013012546 also a food emulator or artificial produce sensor system is disclosed, which aims to replicate a produce's temperature behaviour. The artificial produce sensor system comprises a housing in form of a protective covering sealing with integrated non-perishable, substantially solid material (wax) formed as a block with predetermined mass and shape, wherein the non-perishable material, in combination with the predetermined mass or size, has a temperature retention property similar to a perishable product. At least one temperature sensor is placed in the core of the artificial produce, able to read out core temperatures. The connection between the integrated sensors and an external temperature monitor can be reached either by wire or wireless. All efforts brought an improved simulation of real fruits, but it still does not provide a sufficiently realistic representation of what happens with horticultural produce in the cargo.

The aforementioned artificial produce sensor systems are composed of a simple housing, with cylindrical or square sectional area, in which a kind of filling is placed to provide some thermal inertia and similar thermal conductivity as the food. These simulators neither account for the exact size, three-dimensional shape, surface texture and internal composition of the food (fruit tissue, rind, pit) nor does the filling match all the thermal properties of real food, for example of a specific fruit species. As such, the thermal response of the sensors (conduction, convection, radiation) thereby, cannot fully match that of real produce, by which the sensors used do not provide sufficiently representative fruit core temperature data for monitoring cold chains. The sensor system can also not be directly placed inside a packaging container with fruits and vegetables as it is not made out of food-grade contact materials.

### DESCRIPTION OF THE INVENTION

The object of the present invention is to create an artificial horticultural product, including a sensor system. This product enables to monitor the fruit's thermal behaviour throughout the cold chain in a more realistic way than currently available, including core and surface temperature measurements, by providing an optimized simulation of thermal behaviour of real produce during cooling, refrigerated transport and cold storage of real horticultural produces. Next to core temperatures and surface temperatures, relative humidity measurements are also possible, in order to assess the risk of surface condensation and microbial activity.

To closely match the cooling behaviour of real fruit, a biomimetic approach is pursued which tries to reproduce a real fruit as close as possible. In contrast to the prior art, the size, 3D shape details, surface texture, colour, internal composition (fruit tissue, rind, pit) and all thermal properties (density, specific heat capacity, thermal conductivity, freezing temperature) of the artificial produce are carefully tuned to match those of the horticultural produce species (and cultivar) of interest. To this end, a special type of housing and filling are designed. The filling has a similar composition as real fruit (namely water, carbohydrates, air). The housing can be compartmentalised to hold different fillings. This biomimetic approach leads to a product that reacts thermally very similar to a real produce or fruit, with respect to conduction inside the product, convective heat removal from the product and radiation exchange at the product surface. Thereby, realistic core and surface temperature measurements can be performed.

Additional advantages of the disclosed artificial produce sensor system are that (1) it is a stand-alone unit, which is wireless (no external cabling or temperature loggers but integrated, self-powered data loggers with a built-in temperature sensor) and reusable with autonomy of several years and (2) it is made out of food-grade materials or is coated with them. As such, the artificial fruit does not affect the airflow field and cooling behaviour of surrounding produce, and it can be packed directly with the fresh produce. These advantages enable straightforward installation and retrieval of artificial fruit in a commercial setting at multiple locations in the cargo, to identify the heterogeneity inside a carton or pallet. These artificial fruit can accompany the cargo throughout the entire cold-chain journey, hence avoiding additional handling in between.

Another object of the subject matter of the invention is to provide a manufacturing method for an artificial horticultural produce sensor system, leading to a more realistic housing and filling composition.

### BRIEF DESCRIPTION OF THE DRAWINGS

A preferred exemplary embodiment of the subject matter of the invention is described below in conjunction with the attached drawings.

FIG. 1 shows a perspective exploded view of a partly cutted section through the housing of an artificial produce, while

FIG. 2 shows a partly cutted section through a closed housing partly filled with a gel-like filling composition, where the outer shape of the artificial produce is mimicking a sphere-like produce.

FIG. 3 shows one shell of an artificial product replicating a pear fruit with filled housing, wherein the outer shell comprises a cavity for a data logger.

FIGS. 4 are showing the cooling behaviour of real and artificial produces, here of apple fruit from numerical simulations, wherein core and surface temperatures are depicted.

### DESCRIPTION

This invention concerns an artificial or synthetic horticultural product 1 in form of a sensor system 1, representing a fruit or vegetable.

This artificial horticultural produce 1 comprises a multi-compartment housing 10, a fastening system to (dis)assemble the housing 10, a biomimetic filling 104, and integrated, self-powered data loggers, comprising built-in temperature sensors 1010 1020. The temperature sensors 1010, 1020 allowing monitoring of core and surface temperature history in cold chain operations, by making use of integrated, self-powered data loggers. The shape and thermal properties of the artificial produce are carefully tuned so the synthetic produce 1 is reacting the same as the fresh fruits or vegetables of interest. The integrated temperature loggers are small, robust and wireless, with autonomy of several years.

Housing

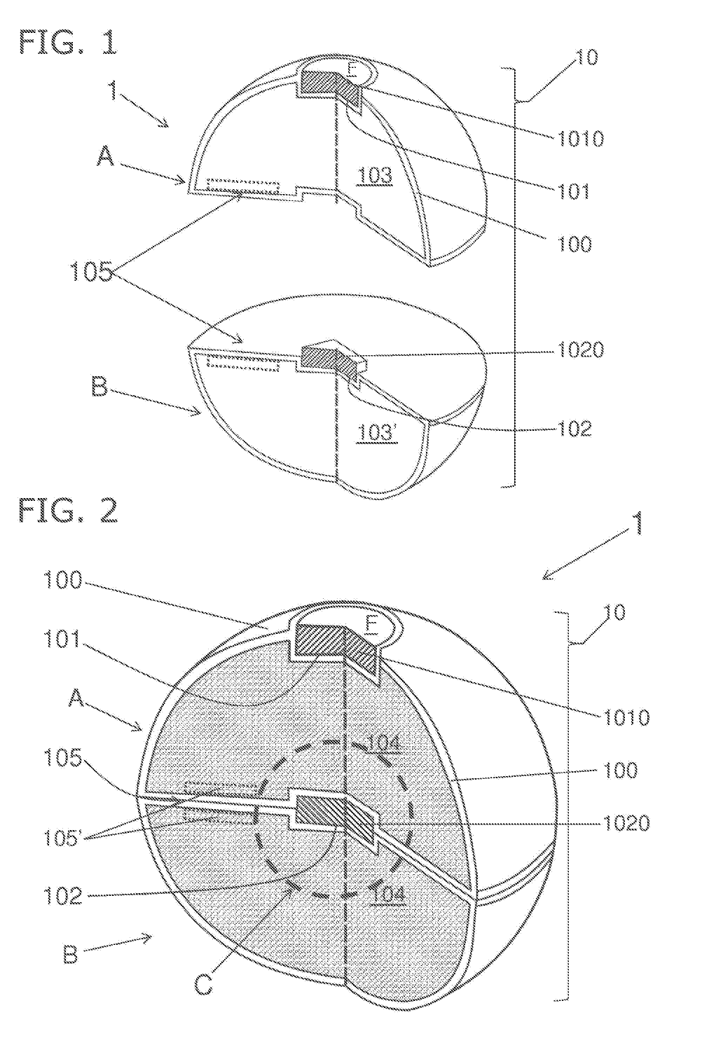

As shown in FIG. 1, the artificial or synthetic horticultural produce 1 in form of a sensor system 1, comprises a housing 10 with a multiplicity of shells A, B, in particular hollow shells A, B. Here two half-shells A, B with walls 100 are forming the housing 10. Both shells A, B can be attached to one another, building a closed housing 10 of the artificial horticultural produce sensor system 1. The housing 10 consists of at least two parts as it has to be filled with the biomimetic filling 104 later on, so this is required for manufacturing purposes of the artificial or synthetic horticultural produce 1. These shells A, B do not have to be of equal size and can also consist of a container with an opening, defined by shell A, which is sealed with a small adequate plug, which could be defined as shell B.

The thin walls 100 of the housing are composed of a plastic, such as acrylic or polyamide, and mimic the exterior size, 3D shape, surface texture of fruits or vegetables of interest, to a certain degree of detail.

For simplification, the figures here only show smooth surfaces. In practice the surface texture can be adapted, to the required degree of detail, to the produce to be simulated. In addition, the housing can also be compartmentalised to include interior composition details if the fruit is composed out of materials with different composition (tissue versus pit). The 3D shape and size of the fruit species (and cultivar) of interest can be chosen in two ways, so that it is representative for an average fruit of the species (or cultivar) or so that it mimics a single fruit of interest.

The shells A, B are hollow forming chambers 103, 103′ which are filled with a thermo-mimetic filling. The first shell A forms a recess 101 for surface data logger in an area near to the outer surface of the housing 10. Both shells A B forms a recess 102 for core data logger in an area later forming the core of the closed housing 10 respectively of the artificial horticultural produce 1. The artificial produce 1 can also be composed of a hollow shell A with one internal space and a plug, via which the thermal filling is inserted in the housing 10.

The chambers 103, 103′ of the hollow shells A, B of the housing are manufactured watertight in order to avoid water migration from the filling to the outside, leading to dehydration and shrinkage of the filling. The housing is given the same color and radiative properties (emissivity) as the fruit of interest, for example by painting.

Data Logger

In the cavity 101 a first data logger 1010 will be placed, which is able to measure the surface temperature and, if requested, the relative humidity (RH) of the ambient air in the vicinity of the artificial produce 1. To not disturb the air flow around the artificial fruit 1 and neighbouring fruits, the cavity 101 of the surface data logger can be disposed counter-sunk in the wall 100 of the first shell A and the depth of the cavity 101 has to be designed accordingly. The surface F of the surface data logger 1010, pointing outward the housing 10, has to have optimum contact to the ambient air surrounding the sensor system 1. The air flow around the housing 10 should be undisturbed and interaction between air flow and the surface data logger 1010 should be minimized, by mounting the data logger flush with the wall 100. The surface data logger 1010 is directly accessible for programming and data readout without disassembling.

A core data logger 1020 is arranged in the cavity 102 of the second shell B. For the logger to monitor a realistic core fruit temperature of produce, the core data logger 1020 has to be placed in the centre area C. This core data logger 1020 can be easily accessed by disassembling the shells A, B.

The data loggers 1010, 1020 used are small, wireless, stand-alone, self-powered data loggers with built-in temperature (and possibly RH) sensors, such as iButtons® or other commercially available systems. Usable data loggers are well known and their electronic structure is explained elsewhere. These small loggers contain an internal battery, which has an autonomy of several years depending on how intensively it is used. They can be programmed with respect to their logging interval and read out after each mission without expert knowledge, where a few 1000 data points can be logged during one mission.

In the core area of the sensor system 1, the second data logger 1020 only measures the temperature of the artificial produce 1.

At the surface of the sensor system 1 with contact to the ambient air, the first data logger 1010 measures the surface temperature and, if required, also relative humidity, depending on the type sensor that is used.

These autonomous data loggers 1010, 1020 are installed in a permanent context in order to monitor both produce core and surface temperature (and RH). The cross-sectional area of used loggers can be circular or polygonal as indicated in FIG. 1.

Optionally, for enhanced usability the sensors may be integrated in a single logger system that can be read out via a wireless data connection or via a central data connection at the surface or any other well-accessible location of the artificial fruit. Furthermore, currently logged data values may be shown in real time using a display at the fruit surface.

Fastening System

A fastener 105, comprising fastening means attached to or formed to the first and the second shell A, B, is indicated in FIG. 1. Due to the fastener 105 the artificial produce 1 can be easily disassembled and it allows easy access to the logger 1020 in the core of the artificial produce 1. For an integrated sensor/logger system with wireless readout without disassembly for example, such a fastening and disassembly system is optional and not necessarily required, as the sensor system can be installed permanently during manufacturing.

In FIG. 2 the fastening means 105′ are indicated in dotted lines as magnetic inlays in each shell A, B, leading to a simple fastening by magnetic forces, when first and second shell A, B are brought close together. With this setup no tools are required for assembling and disassembling. Other fastening means, for example internal and external threads are also possible to be formed at the shells A, B.

Filling composition

The chambers 103, 103′ of the hollow shells A, B are manufactured fluidtight and are filled with a water-based gel-like filling composition 104 for simulating the pulp/tissue of a fruit or vegetable. The filling composition 104 can be defined as a pulp/fruit-tissue simulant. In order to fill the shells A, B with the filling composition 104, the housing 10 respectively the walls 100 of the shells A, B have openings which can be closed (permanently) by plugs. Neither the openings in the chambers 103, 103′ nor the plugs for closing are depicted in the figures.

The filling composition 104 is a water-based gel-like material, with thermal properties that are tuned to be similar to real fruits and vegetables, namely similar thermal conductivity, density, heat capacity and freezing point. The filling composition 104 is built-up depending of the fruit species (and cultivar) of interest. The main idea behind the filling is that it is composed out of the same materials as real fruit, namely water, carbohydrates and air.

The basis of the filling composition 104 comprises a water-carbohydrate mixture. In particular water-soluble carbohydrates are used e.g. disaccharides, such as sucrose. Since carbohydrates are added to the water, the freezing point drops below 0° C., as with real fruit. Thereby, freezing at sub-zero air temperatures, which are often applied in the cold chain, is avoided. By changing the water-carbohydrate mixture, different fruit species or cultivars can be mimicked. The water-carbohydrate composition for many types of horticultural produce is available from literature. As such, the filling of the shell can be directly obtained from tabulated data for a certain type of fruit and does not need to be determined explicitly.

An amount of a filler is added to the gel-like composition 104 to account for the air porosity of the intercellular air spaces. The filler comprises small particles of a light, air-filled material with closed porosity, for example expanded polystyrene particles. The porosity can also be obtained from literature, as it has been determined for many types of food.

A gelling agent or thickening agent, such as carrageenan or agar-agar, is used to immobilize the liquid water-carbohydrate mixture. This avoids natural convective flow of the filling composition 104 inside the shell due to temperature gradients and also mixing of the liquid due to shaking during transport, which would alter the internal heat transfer.

These resulting gel-like composition 104 has a gelling temperature around 30-70° C. These gels can be made thermoreversible with a melting temperature of about 50-90° C., so the gel can be removed from the housing if necessary.

In contrast to previous artificial produce attempts, the present invention is the first to capture the full thermal behaviour in a realistic way by reproducing as close as possible a real fruit of a specific species (and cultivar), in terms of size, 3D shape details, surface texture, colour, internal composition (fruit tissue, rind, pit) and all thermal properties (density, specific heat capacity, thermal conductivity, freezing temperature)

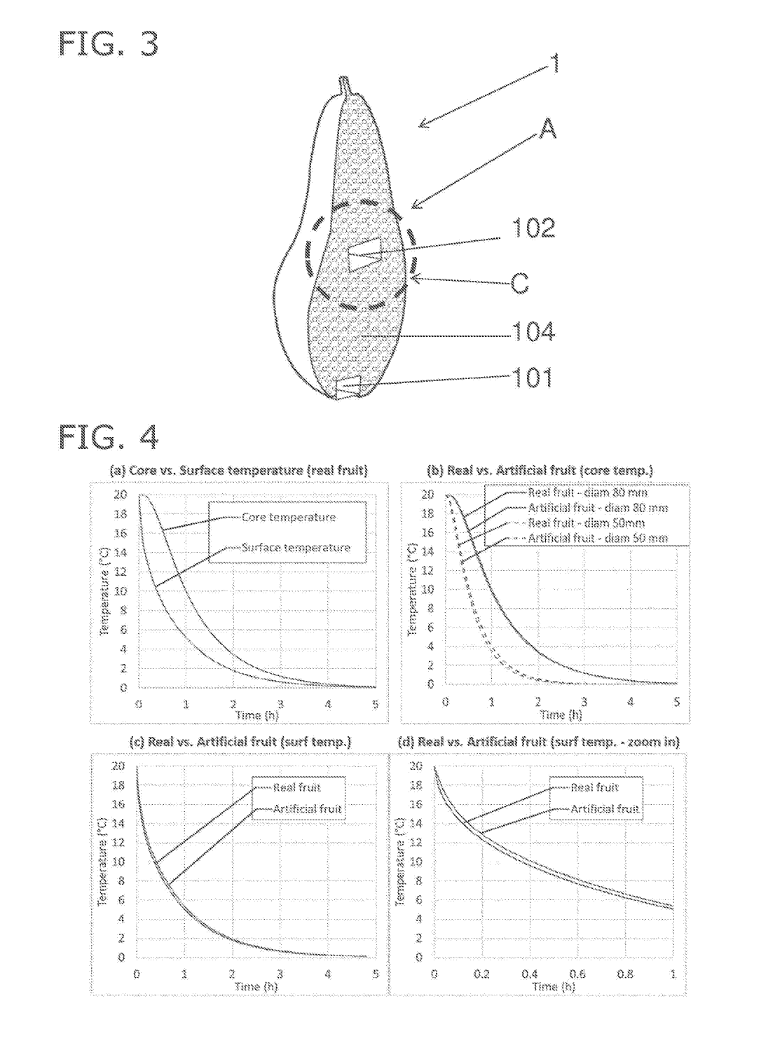

As depicted in FIG. 3, the form of the housing 10 respectively of the shells A, B is adapted to the produce to be simulated and the surface texture of the outer surface also. FIG. 3 depicts one half of a pear, while the shell A has one recess 102 in the area of the core C and one recess 102 in the wall 100 in the vicinity of the outer surface. Again the interior of shell A is filled with the filling composition 104.

Manufacturing Method and Use/Installation in cargo

For manufacturing of artificial horticultural produce in the form of a sensor system the following steps are necessary:

Production of the Housing

In order to construct the housing, non-destructive imaging (surface laser scanning, X-ray imaging, MRI) is used to obtain the size, three-dimensional shape, surface texture and internal features (such as pit or stone) of the target fruit species (and cultivar) of interest. Advanced image processing is used to segment the 3D images and extract the digital 3D surface information by reverse engineering.

This 3D surface information serves as a basis for constructing the full CAD model of the housing 10, namely the outer contours of the shells A, B. The outer surface contour is of primary interest but also the interior composition details can be inferred from such imaging if relevant, such as the size and shape of the stone for mango fruit or the thickness of the rind for orange fruit.

A single fruit can be used to obtain the 3D surface information but also multiple fruit can be scanned to obtain an average fruit shape. To this end, shape description methods can be used to extract an average 3D surface contour from a batch of individual fruit shapes. This custom-made CAD model is then manufactured via rapid prototyping based on additive manufacturing techniques, such as selective laser sintering (SLS) or 3D printing. Note that also simpler shapes can be used as a housing, such as a sphere.

Additive manufacturing is most suitable for production of artificial produce 1 with a complex shape and/or surface details in small quantities. Other manufacturing techniques can also be applied, such as injection moulding, but are less economically viable for small quantities. If necessary, different compartments in the chambers 103, 103′ with different filling composition 104 can be incorporated if a produce has zones with different thermal properties (e.g. large pit in mango, air space with paprika). The chambers 103, 103′ of the housing 10 can be compartmentalised to hold different filling compositions 104 to mimic interior composition differences within produce. This biomimetic approach leads to a product that reacts thermally very similar to a real produce or fruit, with respect to conduction inside the product, convective heat removal from the product and radiation exchange at the product surface. Thereby, realistic core and surface temperature measurements can be performed.

In contrast to previous artificial fruit attempts, the present invention is the first to capture in detail the actual three-dimensional (average or individual) shape and surface texture of any type of horticultural produce, by relying on reverse engineering and rapid prototyping.

The housing is made watertight so no moisture diffuses out of the gel mixture, leading to its dehydration. The outer surface of the housing is given a food-grade coating, which has similar radiative properties as the fruit of interest.

Filling

The internal composition of the fruit is tuned to mimic that of the real fruit species of interest. To this end, a water-carbohydrate mixture is used in which small particles of a light, air-filled material with closed porosity are included in suspension to account for the porosity of the intercellular air spaces in fruit. A specific advantage is that the fruit composition details can be inferred directly from tabulated data so do not have to be explicitly measured.

Assembly

First the housing is designed and manufactured. Then it is filled with the filling composition 104. An appropriate concentration of gelling agent is critical to make sure the light micro-particles maintain evenly distributed in suspension in the gel during the filling of the artificial fruit, but that on the other hand still allows easy injection of the thermal filling material into the housing. If necessary, preservation agents are added in the mixture, to avoid microbial degradation over longer time periods.

Afterwards, two self-powered data loggers with a built-in sensor are integrated in the artificial fruit.

Use in cold chain applications

A critical aspect of the present invention is its user-friendly setup, reuse and data readout, which makes it attractive for commercial R&D cold-chain applications.

At first use, the logging interval of the iButton® loggers 1010, 1020 needs to be set in the provided software by placing the iButton® on the receptor. The core iButton® is easily accessed by just pulling the two parts A, B of the shell apart, and the surface iButton® is directly accessible. After programming, the artificial produce or fruit is closed by the magnetic contacts 105′ and is ready to be used.

Afterwards, the artificial fruit is placed inside the packaging at the desired position in a box (center, edge), and the packaging is closed and palletized. The artificial produce 1 goes through the entire cold chain, or a single unit operation and is retrieved afterwards. The data is read out using the aforementioned procedure.

Application area

Due to the fact, that an artificial fruit is used instead of a real fruit with data loggers, much longer measurements are possible (i.e. months). In addition, beside the core temperature, the surface temperature and even relative humidity are measured (depending on the sensor used at the outside surface). As the artificial produce 1 is a stand-alone unit, it does not affect the airflow and cooling behaviour of surrounding produce in the same storage container in any other way as real produce would do.

The artificial produce 1 is wireless and can be reused many times. This sensor system 1 can be packed directly with the fresh produce as the artificial produce 1 respectively housing 10 has a food grade contact coating. Multiple of them can be easily installed in the cargo. That way, the artificial produce can travel throughout the entire cold-chain journey without additional handling in between cold chain operations.

The artificial produce 1 respectively the sensor system 1 provides a new and more realistic way to monitor the temperature history of the fruit core and its surface along an entire cold chain at multiple locations in the cargo in commercial settings. Such information on the thermal behaviour of the cargo is of direct interest in many cold-chain applications.

It can be used to predict fruit quality or remaining shelf-life. Product temperature can also be linked to the respiratory activity, ripening rate and the efficacy of pest disinfestation by cooling. In addition, the heterogeneity in cooling can be identified at different levels of detail since several fruit can be placed inside a box, a pallet or a cargo. As such, critical points such as respiration-related hot spots can be unveiled. The hygrothermal conditions at the surface can be used to estimate the risk on surface condensation and microbial activity.

### INDUSTRIAL APPLICATION

R&D sections in the cold chain industry (precooling, transport in refrigerated containers and trucks, storage in cold rooms) can benefit from the present invention for similar reasons. The efficacy of new cooling protocols or stowing strategies (intermittent heating and ventilation, cooling unit control, ambient loading) can be evaluated faster, at higher spatial resolution and throughout the entire chain.

In addition, wholesalers and retailers (e.g. Tesco, Wallmart, Coop) are typically interested in exploring new cold chain pathways with a lower carbon footprint. In this context, such sensors could also be used to provide clarity in claims of retailers to producers regarding non-satisfactory product quality, as the loggers can remain inside the packaging all the way up to the retailers.

Feasibility and performance of artificial produce in form of sensor system

To illustrate the feasibility of the artificial produce 1 to accurately mimic surface and core fruit temperatures, compared to real fruit, numerical simulations were performed and are depicted in FIG. 4. For simplicity, a spherical fruit shape was taken. Forced convective cooling of this artificial fruit 1, initially at 20° C., to 0° C. was simulated and compared to that of a real fruit. Representative thermal properties of real fruit (apple) and of all components of the artificial fruit 1 were used in the heat transfer simulations. The artificial fruit 1 was filled with a representative water-carbohydrate-air mixture.

In FIG. 4a, the large difference between surface and core temperatures for a real fruit are indicated. This difference is important, amongst others as governmental organisations (USDA, PPECB) use the core temperature—not the surface temperature—to decide upon the quality of the cargo.

In FIG. 4b, the core temperature of a real fruit is compared to that of the artificial fruit. A very similar behaviour is found, even for small fruit diameters, corresponding to a mandarin for example.

In FIG. 4c-d, the surface temperature measured by the iButton® also shows a very good agreement with that of the real fruit. They differ a bit in the first stage of cooling, due to the difference in thermal properties of the iButton®, compared to real fruit.

### LIST OF REFERENCE NUMERALS

1 artificial or synthetic horticultural produce/sensor system
10 housing
A first shell
B second shell
C area of core
   100 wall
   101 recess /cavity for surface data logger
      1010 surface data logger /humidity and T data logger
      F surface of first data logger
   102 recess/cavity for core logger
      1020 core data logger/T data logger
   103, 103′ chamber
   104 filling composition/fruit pulp simulants
      water-carbohydrate mixture
      gelling agent (e.g. carrageenan)
      filler (expanded polystyrene particles)
   105 fastener
   105′ magnetic means

1. An artificial produce in form of a sensor system, comprising a housing with at least one shell wherein at least one data logger for temperature measurement is placed in an area of a core of the housing and a pulp simulant is integrated in the housing of the artificial produce, wherein
a form of the at least one shell and a surface texture of an outer surface of the at least one shell replicates a form and surface texture of real produce to be simulated and
the at least one shell forms at least one fluidtight chamber accessible from outside by at least one opening in the at least one shell with plugs with each of the at least one opening closable by a plug, wherein the at least one chamber is filled with the pulp simulant in form of a gel-like filling composition comprising a water-carbohydrate mixture and a gelling agent showing similar thermal conductivity, density, heat capacity and freezing point as the pulp of the produce to be simulated.

2. The artificial produce according to claim 1, wherein the gel-like filling composition comprises an amount of a filler in form of small particles of a light, air-filled material with closed porosity to mimic air porosity inside horticultural produce.

3. The artificial produce according to claim 2, wherein the filler comprises expanded polystyrene micro-particles.

4. The artificial produce according to claim 1, wherein the water-carbohydrate mixture is a mixture of water and a soluble carbohydrate, such as the disaccharide sucrose.

5. The artificial produce according to claim 1, wherein the gel-like filling composition has a gelling temperature between 30-70° C. and is thermoreversible with a melting temperature between 50-90° C.

6. The artificial produce according to claim 1, wherein at least one additional data logger for at least one of temperature and humidity measurements is arranged in the housing disposed counter-sunk in the wall of the at least one shell in the area of the surface of the shell pointing outward the housing such that the surface has contact to the ambient air surrounding the artificial produce.

7. The artificial produce according to claim 1, wherein the at least one shell comprises first and second shells connectable by fastening means attached or formed at each shell.

8. The artificial produce according to claim 7, wherein the fastening means are magnetic inlays.

9. The artificial produce according to claim 1, wherein the outer surface of the housing is given the same colour and radiative properties, namely emissivity as a fruit of interest, for example by painting.

10. The artificial produce according to claim 1, wherein the housing has a food grade contact coating by which it can be packed directly with real produce or fruits in a commercial context.

11. A method for manufacturing an artificial produce comprising steps of:
forming a housing in form of at least one shell, having at least one fluidtight chamber, wherein an outer shape of the housing simulates 3D shape, surface texture and internal features of a horticultural produce to be simulated, where the at least one shell includes an opening closeable with a plug,
using a gel-like filling composition having at least a water-carbohydrate mixture and a gelling agent showing similar thermal conductivity, density, heat capacity and freezing point as pulp of the horticultural produce to be simulated, filling of the at least one chamber of the at least one shell with the filling composition and fluidtight closing with the plug,
assembling of at least one temperature data logger in a core area and a temperature and humidity data logger disposed in a wall of the least one shell in an area of a surface of the shell, wherein the temperature and humidity data logger is pointing outward the housing such that the surface of the temperature and humidity data logger has contact to the ambient air surrounding the artificial produce.

12. The method according to claim 11, wherein the at least one shell comprises first and second shells connected by fastening means attached or formed at each shell.

13. The method according to claim 11, wherein the filling composition comprises an amount of a filler in form of small particles of a light, air-filled material with closed porosity.

14. The method according to claim 11, wherein the temperature and humidity data logger is disposed counter-sunk in the wall of the at least one shell pointing outward the housing.

15. The method according to claim 12, wherein the fastening means includes magnetic inlays.

16. A housing of an artificial produce with at least one shell, wherein at least one data logger is placeable in a core area of the housing and a pulp simulant is integrateable in the housing, wherein
a form of the shell and a texture of an outer surface of the at least one shell is replicating form and surface texture of real produce to be simulated, the at least one shell forming at least one chamber accessible from the outside by at least one opening in the at least one shell which are closeable with plugs, wherein the at least one chamber is fillable with a gel-like filling composition and at least one core data logger is placeable in a recess for data logger while at least one surface data logger is placeable in a recess in contact with the outer surface of the housing respectively the at least one shell, so that, a surface of the at least one surface data logger is placeable with access to ambient air outside the housing.

17. The housing according to claim 16, wherein the housing comprises a container as a first shell with an opening, which is sealable with a plug, defined as a second shell.
